(19) United States
(12) Patent Application Publication
(10) Pub. No.: US 2026/0309688 A1
(43) Pub. Date: Oct. 8, 2026

(54) GAS SENSOR FOR SKIN GAS AND GAS SENSOR SYSTEM FOR SKIN GAS

(71) Applicant: NISSHA CO., LTD., Kyoto-shi (JP)

(72) Inventors: Itsuo KOJIMA, Kyoto-shi (JP); Junichi SHIBATA, Kyoto-shi (JP); Aki UESAKA, Kyoto-shi (JP); Kana KOBAYASHI, Kyoto-shi (JP); Kazuya OKI, Kyoto-shi (JP)

(21) Appl. No.: 19/575,174

(22) Filed: Mar. 23, 2026

(30) Foreign Application Priority Data
Apr. 8, 2025 (JP) 2025-063508

(51) Int. Cl. G01N 33/00; G01D 11/24; G01N 1/02; G01N 33/497

(52) U.S. Cl. CPC G01N 33/0073; G01D 11/245; G01N 1/02; G01N 33/497; G01N 2001/028

(57) ABSTRACT

A gas sensor for skin gas includes a gas-sensing element, a housing, a pump, a gas inlet, and an exhaust outlet. The gas-sensing element detects a specific component contained in skin gas generated from a surface of skin. The housing includes an accommodating space having a volume in which the gas-sensing element detects the specific component and housing the gas-sensing element. The pump is arranged on a first surface of the housing to exhaust air in the accommodating space. The gas inlet supplies outside air to the accommodating space and is provided on a second surface of the housing facing the first surface. The exahust outlet exhausts air in the accommodating space in response to driving of the pump.

This application claims priority to Japanese Patent Application No. 2025-063508 filed on Apr. 8, 2025 with Japan Patent Office. The entire disclosure of Japanese Patent Application No. 2025-063508 is hereby incorporated herein by reference.

### BACKGROUND

### FIELD OF THE INVENTION

The present invention relates to a gas sensor for skin gas and a gas sensor system for skin gas for detecting a specific component contained in skin gas generated from a surface of skin.

### BACKGROUND INFORMATION

It is known that a detection result of a specific component such as acetone contained in skin gas generated from a surface of skin can be applied to health care and health diagnosis. JP 2019-070612 A discloses a skin gas measurement device capable of measuring a specific component such as acetone contained in skin gas. The skin gas measurement device disclosed in JP 2019-070612 A measures a concentration of a specific component contained in skin gas of a user by contacting a sole of the user’s foot.

### SUMMARY

In the skin gas measurement device of JP 2019–070612 A, a measurement is started when a limit switch detects movement of a housing itself accompanying an action of pressing skin. Since this configuration requires a space sufficient for the housing to move, it has been difficult to miniaturize an enclosure for housing the skin gas measurement device. In addition, when measuring a specific component intermittently, it has been necessary to provide an interval longer than a certain time in order to replace air in a skin gas collection space.

One object is to provide a gas sensor for skin gas and a gas sensor system for skin gas which can suppress an increase in a detection interval even when detecting a specific component intermittently while suppressing an increase in size of a device.

Hereinafter, a plurality of aspects will be described as means for solving the problem. These aspects can be combined in any manner if necessary.

A gas sensor for skin gas according to an embodiment of the present disclosure includes a gas-sensing element, a housing, a pump, a gas inlet and an exhaust outlet. The gas-sensing element is configured to detect a specific component contained in skin gas generated from a surface of skin. The housing includes an accommodating space having a volume in which the gas-sensing element is configured to detect the specific component and housing the gas-sensing element. The pump is arranged on a first surface of the housing to exhaust air in the accommodating space. The gas inlet is configured to supply outside air to the accommodating space and is provided on a second surface of the housing facing the first surface. The exhaust outlet is configured to exhaust air in the accommodating space in response to driving of the pump.

With the gas sensor mentioned above, the pump includes a positive displacement pump of a flat shape along the first surface of the housing. The housing further includes an air supply port provided on the first surface to supply air supplied from the pump to the accommodating space.

With any one of the gas sensors mentioned above, the gas-sensing element, the gas inlet, and the air supply port are arranged at positions not overlapping each other in a thickness direction of the housing.

With any one of the gas sensors mentioned above, the gas inlet and the exhaust outlet are provided on different surfaces of the housing.

With any one of the gas sensors mentioned above, the volume of the accommodating space is from 170 mm³ or more to 1,000 mm³ or less.

With any one of the gas sensors mentioned above, the housing has a concave portion on an outer surface of the second surface. The gas inlet is arranged on a bottom surface of the concave portion.

A gas sensor system for skin gas according to an aspect of the present disclosure includes any one of the gas sensors mentioned above, and a control unit configured to control the gas sensor.

The control unit includes a detection information acquisition unit, an instruction unit, a pump drive unit, and a gas information acquisition unit. The detection information acquisition unit is configured to acquire information for driving the pump inputted from outside. The instruction unit confgured to control driving of the pump and acquisition timing of gas information of the gas-sensing element based on an input of the detection information acquisition unit. The pump drive unit is configured to control driving of the pump based on an instruction of the instruction unit. The gas information acquisition unit is configured to acquire gas information of the gas-sensing element based on an instruction from the instruction unit after completion of driving of the pump.

With the gas sensor system mentioned above, the instruction unit is conifgured to instruct the pump drive unit to start driving the pump, configured to instruct the gas information acquisition unit to start detection, and configured to start detection after a predetermined time after completion of driving the pump.

With any one of the gas sensor system mentioned above, the detection information acquisition unit is configured to detect that the gas sensor is attached to a user.

According to a gas sensor for skin gas and a gas sensor system for skin gas in accordance with the present disclosure, an increase in a detection interval is suppressed even when a specific component is detected intermittently, while an increase in size of a device is suppressed.

### BRIEF DESCRIPTION OF THE DRAWINGS

FIG. 1 is a plan view of a gas sensor according to an exemplary embodiment of the present disclosure;

FIG. 2 is an exploded perspective view of a gas sensor according to an exemplary embodiment of the present disclosure;

FIG. 3 is a cross-sectional view of a gas sensor according to an exemplary embodiment of the present disclosure;

FIG. 4 is a functional block diagram of a gas sensor system according to an exemplary embodiment of the present disclosure;

FIG. 5 is a control flow executed by a gas sensor system according to an exemplary embodiment of the present disclosure; and

FIG. 6 is a cross-sectional view showing a state of use of a gas sensor according to an exemplary embodiment of the present disclosure.

### DETAILED DESCRIPTION OF EMBODIMENTS

### 1. Gas sensor 1

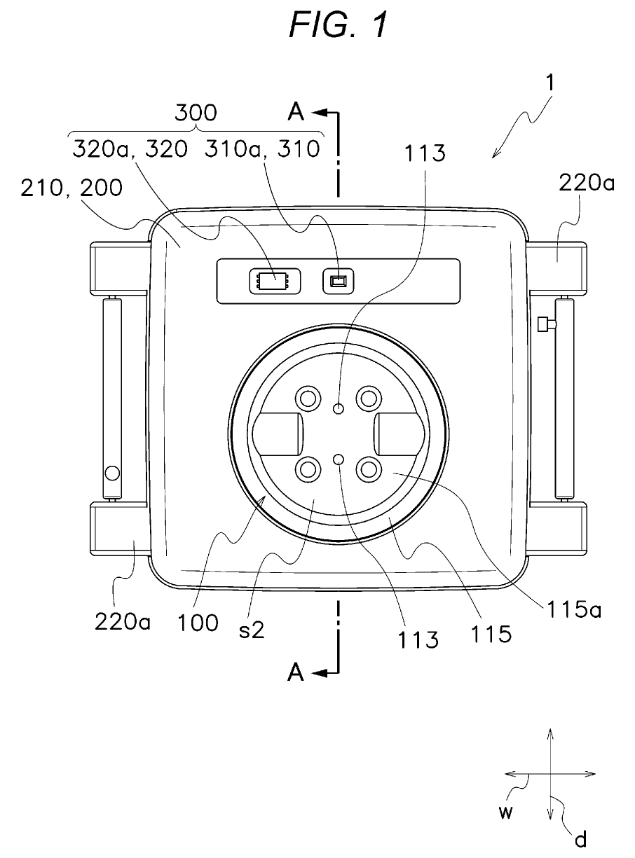

A gas sensor 1 according to the present exemplary embodiment will be described with reference mainly to FIGS. 1-3 and 6. The gas sensor 1 includes a housing 110, a gas-sensing element 120, a pump 130, a detection unit or detector 300, and a control unit or electronic controller 400. The gas sensor 1 is a skin gas sensor for detecting a specific component contained in skin gas generated from a surface of skin.

A specific component to be detected by the gas sensor 1 is, for example, acetone. Acetone is produced as a metabolite in blood by decomposition of body fat. By measuring an amount of acetone contained in skin gas, a user of the gas sensor 1 can use the amount as an index of the user's own health.

Upward and downward directions (UP, DOWN), a height direction h, a depth direction d, and a width direction w used in the following description correspond to the directions indicated by arrows in the figures. Furthermore, the upward and downward directions, the height direction h, the depth direction d, and the width direction w are used in the following description for convenience of description and do not limit a posture of the gas sensor 1 in use.

### 1. Housing 110

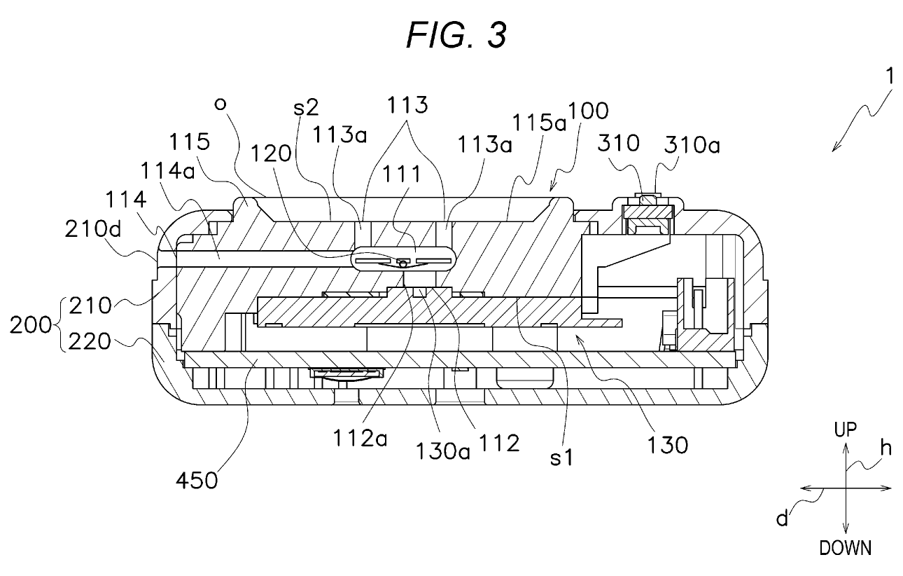

The housing 110 is a member having a structure in which the gas-sensing element 120 can detect skin gas while housing the gas-sensing element 120. The housing 110 includes a first surface s1, which is a surface facing the pump 130, and a second surface s2, which faces outside air or skin of a user. In the present exemplary embodiment, the housing 110 is a flat member extending in a horizontal direction (depth direction d and width direction w). The housing 110 has an accommodating space 111, an air supply port 112, at least one (two in FIG. 3) gas inlet 113, an exhaust outlet 114, and a projection 115.

The accommodating space 111 has volume V in which the gas-sensing element 120 can detect a specific component while storing the gas-sensing element 120. The accommodating space 111 is provided in a hollow shape substantially in the center of the housing 110 and has a flat shape extending in an extending direction of the housing 110. Here, a concentration of acetone gas contained in skin gas is very low at about one twentieth of expired gas. Therefore, the smaller volume V of the accommodating space 111, the better. The accommodating space 111 in the present exemplary embodiment has volume V of, for example, from 170 mm³ to 1,000 mm³. Thus, acetone gas contained in skin gas having a low concentration can be quickly detected.

The air supply port 112 is provided on the first surface s1, and air discharged from the pump 130 is sent into the accommodating space 111 through an air supply path 112a. Although the air supply path 112a in the present exemplary embodiment is provided in a straight line from the air supply port 112, the air supply path 112a may be provided in a curved line in consideration of a wind pressure of the pump. In plan view, it is preferable that the air supply path 112ais disposed so as not to overlap with the gas-sensing element 120. As a result, it is possible to reduce an influence that can be received by wind from the pump 130, such as a drop in temperature of a surface of the gas-sensing element 120.

Each gas inlet 113 is provided on the second surface s2, and skin gas in skin gas collection space sp is sent into the accommodating space 111 via a gas inlet path 113a. Although the gas inlet path 113a in the present exemplary embodiment is provided in a straight line from the gas inlet 113, the gas inlet path 113a may be provided in a curved line from a viewpoint of preventing contamination with foreign matter such as sweat or from a viewpoint of improving gas selectivity. “Improving gas selectivity” as referred to herein is intended to selectively detect acetone having a fast diffusion rate by utilizing a difference in diffusion rates of acetone, ammonia, acetic acid, diacetyl, 2-nonenal, and the like, which are skin gas components. Furthermore, although the housing 110 in the present exemplary embodiment is provided with two gas inlets 113, the number of gas inlets is not limited as long as a specific component can be detected appropriately.

The exhaust outlet 114 is provided on a side surface of the housing 110 and exhausts air in the accommodating space 111 to the outside in response to driving of the pump 130 through an exhaust path 114a. The exhaust outlet 114 in the present exemplary embodiment is preferably provided at substantially the same height as the gas-sensing element 120. Thus, the accommodating space 111 can be made into a thin film. Furthermore, although the exhaust outlet 114 is provided on the side surface on the left side of the drawing among the side surfaces of the housing 110, it may be provided at a position where it is not occluded by the skin 500 of a user during use. A position of the exhaust outlet 114 does not matter as long as the exhaust outlet 114 can appropriately exhaust air in the accommodating space 111 in response to driving of the pump 130.

The projection 115 is a convex portion provided on the second surface s2 and forming a skin gas collection space sp, and is a portion that comes into contact with the skin 500 of a user when the gas sensor 1 is used. The projection 115 is arranged on an outer periphery of the second surface s2 relative to the gas inlets 113 in plan view. The second surface s2 located on an inner periphery of the projection 115 forms a concave portion or recess 115a of the skin gas collection space sp. In other words, each gas inlet 113 is arranged in the concave portion 115a. The gas sensor 1 of the present exemplary embodiment can secure a fixed volume of the skin gas collection space sp because of the configuration of the projection 115 and can prevent each gas inlet 113 from being blocked by the skin 500.

The skin gas collection space sp is provided on the second surface s2 of the housing 110 and is a substantially closed space formed by the projection 115, the concave portion (or planar recess) 115a, and the skin 500 of a user. By providing the skin gas collection space sp, skin gas of low concentration can be efficiently collected.

The material of the housing 110 may be, for example, Teflon (R), polypropylene, ABS resins, polyethylene resins, polyethylene terephthalate, polymethyl methacrylate resins, polyamide resins, polyacetal resins, epoxy resins, polyvinyl chloride, or the like. Although not limited, Teflon (R), which adsorbs less gas than metal, is preferable as the material of the housing 110.

The gas-sensing element 120 detects a specific component contained in skin gas having been introduced into the accommodating space 111. The gas-sensing element 120 outputs a detection result of the specific component contained in skin gas as gas information. The gas sensor 1 has a first gas-sensing element 120a and a second gas-sensing element 120b as the gas-sensing element 120. The first gas-sensing element 120a and the second gas-sensing element 120b are disposed in the accommodating space 111 at positions not overlapping each gas inlet 113 in plan view. Although not limited, in the present exemplary embodiment, The gas-sensing element 120 is a semiconductor gas sensor that preferably detects acetone having a ketone group.

In the present exemplary embodiment, the gas-sensing element 120 and the air supply port 112 are disposed at positions not overlapping each other in a thickness direction (the height direction h) of the housing 110. This prevents occurrence of defects in the gas-sensing element 120, such as damage to the gas-sensing element 120 or deterioration in accuracy of the gas-sensing element 120, when air flowing in from each gas inlet 113 or the air supply port 112 directly hits the gas-sensing element 120.

The pump 130 is disposed opposite to the first surface s1, and air generated from the pump 130 is sent into the accommodating space 111 via the air supply port 112. The pump 130 can be various pumps, such as a positive displacement pump such as a reciprocating pump or a rotary pump, or a non-positive displacement pump such as a centrifugal pump, a propeller pump, or a viscous pump. The pump 130 of the present exemplary embodiment is a positive displacement pump in which a flat diaphragm along the first surface s1 of the housing 110 is movable. Specifically, the pump 130 is a piezoelectric micropump having a piezoelectric element and a diaphragm (both are omitted from the illustration). The pump 130 is controlled by the control unit 400 (described later). The pump 130 feeds air from a blower 130a provided at a position opposed to the air supply port 112. In this way, it is possible to suppress an increase in size of the gas sensor 1.

The pump 130 is arranged on the first surface s1 of the housing 110 so as to discharge air introduced into the accommodating space 111. Specifically, air fed from the blower 130a flows into the accommodating space 111 through the air supply port 112 and the air supply path 112a. Here, since a space on a gas inlet path 113a side is closed by the skin 500, air in the accommodating space 111 is surely discharged through the exhaust path 114a. Since air can be discharged surely by driving the pump 130 in this way, a volume or driving of the pump 130 can be small, and an increase in size of the gas sensor 1 can be suppressed.

### 2. Enclosure 200

The gas sensor 1 may be covered with an enclosure 200. The enclosure 200 includes an upper enclosure 210 and a lower enclosure 220.

The upper enclosure 210 is provided with a first opening 210a, a second opening 210b, a third opening 210c, and a fourth opening 210d. The first opening 210ais an opening for exposing the second surface s2 of the housing 110 upward. The second opening 210band the third opening 210care openings for exposing the detection unit 300 upward. The fourth opening 210dis an opening for exposing the exhaust outlet 114.

The lower enclosure 220 may be provided with band attachment portions 220a on side surfaces to which a band, which is not illustrated, can be attached. Thus, the gas sensor 1 can be attached to a user's arm or the like.

### 3. Detection unit 300

The detection unit 300 detects detection information used by the control unit 400 for determining whether or not a detection process can be started. The detection unit 300 of the present exemplary embodiment includes two sensors, that is, a temperature sensor 310 and an illuminance sensor 320, in order to acquire two pieces of detection information, but the detection unit 300 may be one or more. Detection information of the present exemplary embodiment is temperature information detected by the temperature sensor 310 and illuminance information detected by the illuminance sensor 320.

The temperature sensor 310 is positioned in the second opening 210b. A detection surface 310a of the temperature sensor 310 is exposed from the upper enclosure 210 through the second opening 210b. Similarly, a detection surface 320aof the illuminance sensor 320 is exposed from the upper enclosure 210 through the third opening 210c. The detection surface 310aand the detection surface 320a are at the same or substantially the same height as the projection 115 in the height direction h. Thus, the temperature sensor 310 can set a predetermined temperature associated with use of the gas sensor 1 (for example, around 30°C, which is a surface temperature of skin) as a threshold value, and the illuminance sensor 320 can set a predetermined illuminance associated with use of the gas sensor 1 (for example, darkness of about 3 lx) as a threshold value. By combining a plurality of sensors, malfunction can be reliably prevented.

Although the temperature sensor 310 and the illuminance sensor 320 are used as examples in the detection unit 300 of the present exemplary embodiment, it is sufficient to detect start of use of the gas sensor 1, and any sensor such as an acceleration sensor, a gyro sensor, a capacitance sensor, a light sensor, an atmospheric pressure sensor, a pressure sensor, or a force sensor can be used. Furthermore, the detection unit 300 can detect various signals such as external signals used in combination with or independently of the above-described sensors. The external signals referred to here are, for example, signals input from a switch or the like for instructing driving of the gas sensor 1.

### 4. Control unit 400

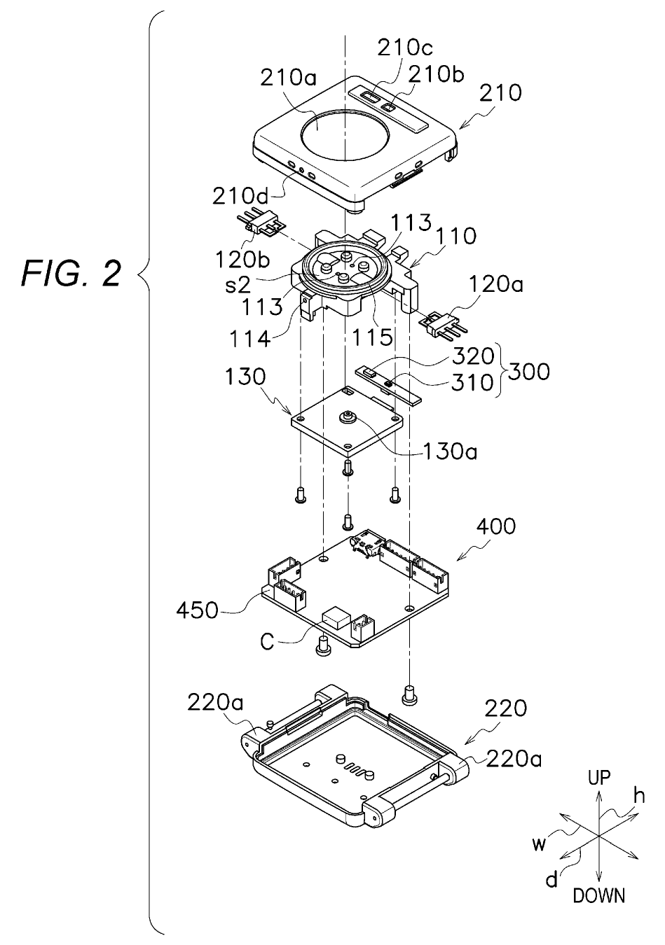

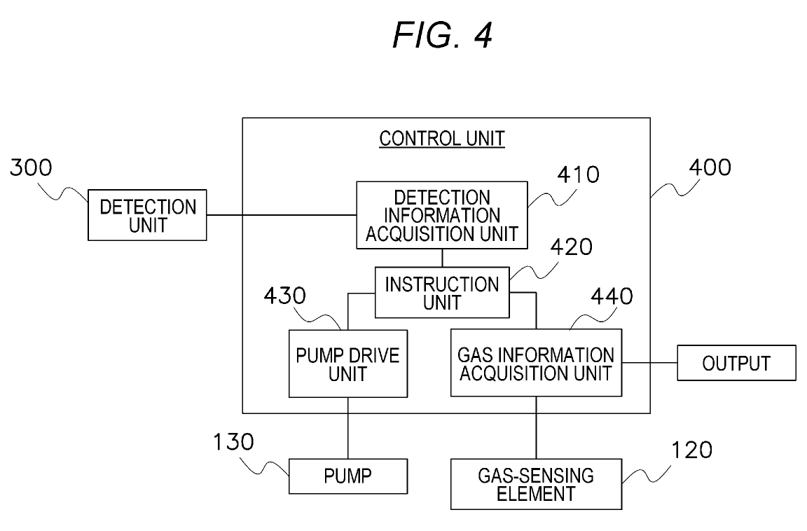

Referring to FIGS. 2 and 4, the control unit 400 controls an entire operation of the gas sensor 1. In particular, a detection process of detecting a specific component contained in the skin gas and a process of controlling the drive timing of the pump 130 are executed. The control unit 400 includes a substrate 450 and a MCU (Micro Controller Unit) or microcontroller C arranged on the substrate 450. FIG. 4 is a functional block diagram of a gas sensor system. The control unit 400 of the present exemplary embodiment includes a detection information acquisition unit 410, an instruction unit 420, a pump drive unit 430, and a gas information acquisition unit 440.

The Micro Controller Unit C may have one or more configurations selected from an arithmetic unit, a storage unit, a communication unit, and the like as required (all of which are omitted from the illustration). Functions of the arithmetic unit are realized by executing a program stored in advance. Functions of the storage unit are realized by reading, for example, a nonvolatile memory or a storage area on a cloud. The functions of the communication unit are realized by, for example, transmitting and receiving information to and from the detection unit 300 or the gas sensor 1 by wired communication, and transmitting and receiving information to and from an external device by wireless communication. The arithmetic unit can write calculation results into the storage unit or read information from the storage unit according to a program. In the illustrated embodiment, the Micro Controller Unit C includes at least one processor having a CPU (Central Processing Unit), a storage device or computer memory and a communication interface for communication, as the arithmetic unit, the storage unit and the communication unit, respectively.

The detection information acquisition unit 410 acquires information for driving the pump 130 inputted from the outside. “Information for driving the pump 130” as referred to herein is, for example, temperature information and illuminance information, which are two pieces of detection information acquired from the detection unit 300. Based on these pieces of detection information, the system detects attachment of the gas sensor 1 to a user.

The instruction unit 420 controls two operations, i.e., driving of the pump 130 and acquisition timing of gas information of the gas-sensing element 120, based on an input of the detection information acquisition unit 410. If necessary, the instruction unit 420 may be connected to an arithmetic unit, a storage unit, or the like so that mutual information can be read. For example, by recording “gas information re-acquisition time” in the storage unit, the instruction unit 420 can start acquisition of gas information after a predetermined time after completion of driving of the pump 130. Further, for example, by computing “pump driving time” in the arithmetic unit, a driving time of the pump 130 can be instructed together with start of driving of the pump 130.

The pump drive unit 430 controls driving of the pump 130 based on an instruction from the instruction unit 420. “Driving of the pump 130” includes, for example, a drive timing of the pump 130, a driving time of the pump 130, a number of times of driving the pump 130, and a driving stop time of the pump 130. The pump drive unit 430 may execute a uniform operation according to an instruction from the instruction unit 420, may execute an operation by reading driving information stored in the storage unit, or may execute an operation based on a calculation result executed by the arithmetic unit.

The gas information acquisition unit 440 acquires gas information of the gas-sensing element 120 based on an instruction of starting detection from the instruction unit 420 after completion of driving of the pump 130. That is, the gas information acquisition unit 440 may acquire gas information immediately after completion of driving the pump 130, or may acquire gas information after a predetermined time elapses after completion of driving the pump 130. The gas information acquisition unit 440 of the present exemplary embodiment acquires a change in a resistance value of the gas-sensing element 120. The acquired gas information may or may not be converted into a numerical value that can be used as gas detection information by, for example, interconnecting with the arithmetic unit.

The gas information acquired by the gas information acquisition unit 440 is output to the outside of the gas sensor 1 via an output unit (not illustrated). Output gas information may be displayed on a display of a device incorporating the gas sensor 1, for example, or output gas information may be stored on a cloud and displayed so that it can be confirmed in a desired application.

### 5. Method of Using Gas Sensor

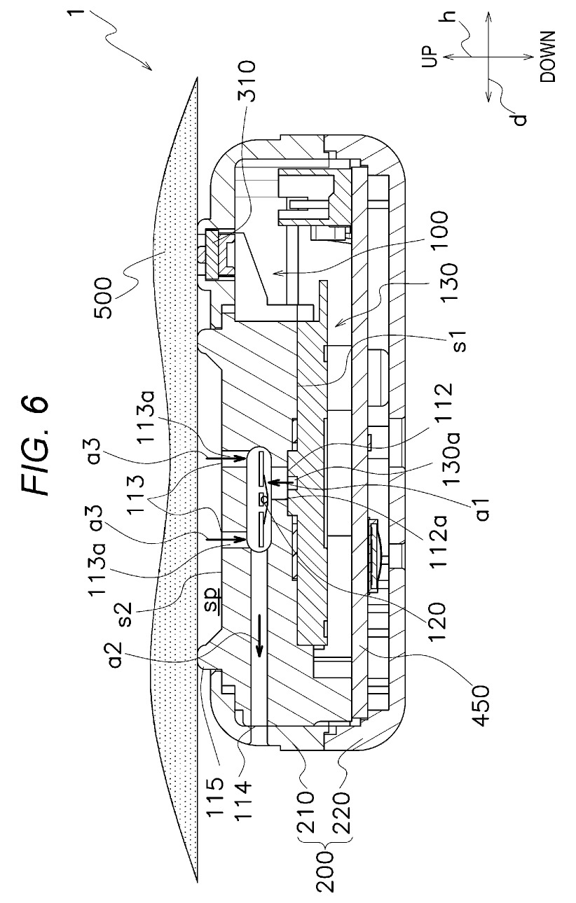

A method of using the gas sensor 1 of the present exemplary embodiment will be described with particular reference to FIG. 1. The gas sensor 1 can be attached to a user’s arm or the like via a band attached to the band attachment portions 220a of the enclosure 200. Upon attachment, the second surface s2 of the housing 100 is closed by the skin 500 (see FIG. 6). As a result, the skin gas collection space sp becomes a substantially closed space, so that skin gas having a low concentration can be efficiently taken into the housing 100.

In addition, it is precisely because the skin gas collection space sp is a substantially closed space, air in the accommodating space 111 can be directed toward the exhaust outlet 114 instead of toward each gas inlet 113 during driving of the pump 130. Thus, air in the accommodating space 111 can be efficiently refreshed, and skin gas concentration in the skin gas collection space sp can be prevented from being diluted. In other words, according to the gas sensor 1 of the present exemplary embodiment, skin gas can be measured more quickly and accurately than before.

In addition, the gas sensor 1 of the present exemplary embodiment can start skin gas detection without using a limit switch for physical driving as in the prior art, so that the enclosure can be miniaturized.

### 6. Gas Sensor System

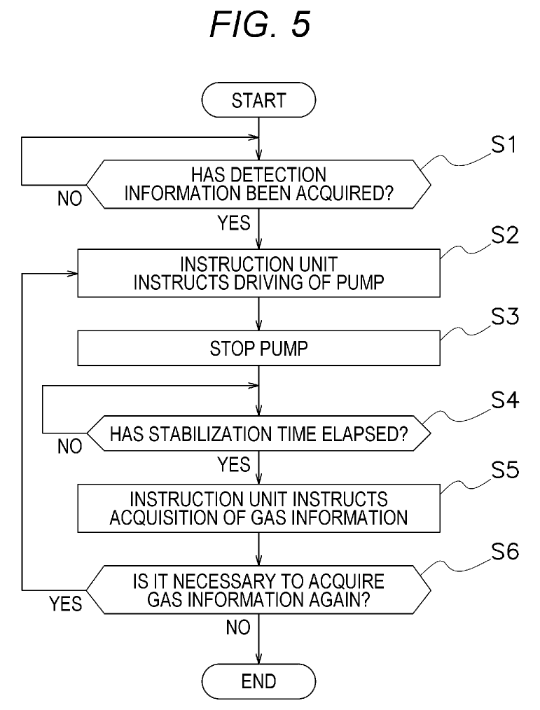

Next, the gas sensor system of the present exemplary embodiment will be described with reference to FIG. 5. FIG. 5 is a control flow executed by the control unit 400.

The control flow of the present exemplary embodiment starts in response to a user starting to use the gas sensor 1. Starting to use the gas sensor 1 includes not only an operation of turning on a main power supply but also an operation of ending a charging state and mounting the gas sensor 1 by the user. When the control flow is started, the system determines whether detection information associated with mounting by the user has been acquired or not (step S1). If detection information has been acquired (YES in step S1), the instruction unit 420 instructs the pump 130 to start driving (step S2). In step S2, the instruction unit 420 may instruct a driving time of the pump 130 together with an instruction to start driving the pump 130. Since the accommodating space 111 of the gas sensor 1 can be refreshed by driving the pump 130, the pump driving time may be regarded as a “refresh time”. The refresh time is, for example, from 1 second to 7 seconds. Since the accommodating space 111 of the gas sensor 1 of the present exemplary embodiment is very small as compared with a gas sensor for exhaled gas, the refresh time can be shortened by twice or more.

On the other hand, if some or all of the detection information cannot be acquired (NO in step S1), the system repeats until all of detection information is acquired or waits until a user’s input is detected. Here, “detection information cannot be acquired” includes that acquired detection information is below a desired threshold value.

Step S2 ends when the pump 130 is stopped (step S3). Step S3 may be performed in response to an instruction from the instruction unit 420 to the pump drive unit 430 in step S2, or the instruction unit 420 may instruct the pump drive unit 430 to stop the pump 130. Here, “in response to an instruction” includes that pump driving ends when an instruction from the instruction unit 420 to the pump drive unit 430 ends.

After the pump 130 is stopped, in the present exemplary embodiment, a “stabilization time”, which is a time for stabilizing the gas-sensing element 120 before acquiring gas information, may also be provided (step S4). The stabilization time is, for example, from 5 seconds to 60 seconds. Thus, a resistance value of the gas-sensing element 120 after air in the accommodating space 111 is refreshed can be stabilized, and more accurate gas information can be acquired.

After the resistance value of the gas-sensing element 120 is stabilized, the instruction unit 420 instructs the gas information acquisition unit 440 to acquire gas information (step S5). Step S5 may determine whether or not the resistance value is stabilized based on gas information from the gas information acquisition unit 440, may be executed after a predetermined time from step S2, or may be executed based on an input from the user. The acquired gas information is transmitted to a device or an application using skin gas information via wired or wireless communication.

After the skin gas information is transmitted to the outside, the user can select whether or not re-acquisition of gas information is necessary at an arbitrary timing or at a timing predetermined by a program (step S6). If YES in step S6, the process returns to step S2, and the above-described operations can be repeated. If NO in step S6, that is, if the user stops using the gas sensor 1, the gas sensor system is terminated.

Specific components to be detected by the gas sensor 1 are not limited to acetone. Specific components to be detected by the gas sensor 1 are, for example, components associated with physical conditions (health, disease, sleep, rest, exercise, etc.), and may be various inorganic components (for example, hydrogen, ammonia, carbon monoxide, carbon dioxide, nitric oxide, hydrogen sulfide, etc.) and various organic components (for example, methanol, ethanol, methane, isoprene, trimethylamine, formaldehyde, acetaldehyde, nonenal, etc.) detectable by the gas sensor 1.

Specific components to be detected by the gas sensor 1 may be not only components derived in vivo but also components derived from outside the body (environmental). The components derived from outside the body are, for example, components released from a surface of skin after respiration or percutaneous absorption of various artificial components contained in a working environment or living environment. Specifically, it is known that these components are released from a surface of skin when exposed to organic solvents (benzene, toluene, etc.), chlorinated solvents, or the like in an office or laboratory handling chemicals.

Furthermore, the gas sensor 1 is not limited to a semiconductor gas sensor as long as it can detect a skin gas component. The gas sensor 1 may be, for example, an electrochemical sensor, a gas heat conduction sensor, a surface acoustic wave sensor, a contact combustion sensor, an optical sensor, or the like.

The exemplary embodiments and modifications of the present invention have been described above. However, the present invention is not limited to these configurations, and various modifications can be made without departing from the gist of the invention. In particular, the plurality of modifications described in the present specification can be combined in any manner as necessary.

What is claimed is:

1. A gas sensor for skin gas comprising:
a gas-sensing element configured to detect a specific component contained in skin gas generated from a surface of skin;
a housing including an accommodating space having a volume in which the gas-sensing element is configured to detect the specific component and housing the gas-sensing element;
a pump arranged on a first surface of the housing to exhaust air in the accommodating space;
a gas inlet configured to supply outside air to the accommodating space and provided on a second surface of the housing facing the first surface; and
an exhaust outlet configured to exhaust air in the accommodating space in response to driving of the pump.

2. The gas sensor for skin gas according to claim 1, wherein
the pump includes a positive displacement pump having a flat shape along the first surface of the housing, and
the housing further includes an air supply port provided on the first surface to supply air supplied from the pump to the accommodating space.

3. The gas sensor for skin gas according to claim 2, wherein
in a thickness direction of the housing, the gas-sensing element, the gas inlet, and the air supply port are arranged at positions not overlapping each other.

4. The gas sensor for skin gas according to claim 1, wherein
the gas inlet and the exhaust outlet are provided on different surfaces of the housing.

5. The gas sensor for skin gas according to claim 1, wherein
the volume of the accommodating space is from 170 mm³ to 1,000 mm³.

6. The gas sensor for skin gas according to claim 1, wherein
the housing has a concave portion on an outer surface of the second surface, and
the gas inlet is arranged on a bottom surface of the concave portion.

7. A gas sensor system for skin gas comprising:
the gas sensor according to claim 1; and
a control unit configured to control the gas sensor, the control unit including
a detection information acquisition unit configured to acquire information for driving the pump inputted from outside,
an instruction unit configured to control driving of the pump and acquisition timing of gas information of the gas-sensing element based on an input from the detection information acquisition unit,
a pump drive unit configured to control driving of the pump based on an instruction from the instruction unit, and
a gas information acquisition unit configured to acquire gas information of the gas-sensing element based on an instruction from the instruction unit after completion of the driving of the pump.

8. The gas sensor system for skin gas according to claim 7, wherein
the instruction unit is
configured to instruct the pump drive unit to start driving the pump,
configured to instruct the gas information acquisition unit to start detection, and
configured to start the detection after a predetermined time after completion of the driving of the pump.

9. The gas sensor system for skin gas according to claim 8, wherein
the detection information acquisition unit is configured to detect that the gas sensor is attached to a user.
